SEQUENCE LISTING (7 sequences)

SEQ ID NO: 1 - DNA, 1272 bp, Equus caballus
  source (1)..(1272): cDNA based on mRNA from equine peripheral blood mononuclear cells
gtctccaagcaagccccattaatcttgcccttggctgcctgctgcaaagacaccaagactactaacatcacactgggctgcctggtcaagggctacttcccggagccagtgaccgtgacctgggatgcagggtcccttaaccggagcaccatgaccttccctgccgtctttgaccaaacctctggcctctacaccaccatcagcagggtggtcgcctcggggaagtgggccaagcagaagttcacctgcaacgtggtgcactcccaggagaccttcaacaagaccttcaacgcatgcatcgtgaccttcaccccacccaccgtgaagctcttccactcctcctgcgaccccggcggcgactcccataccaccatccagctcctgtgcctcatctccgactacacccctggcgacatcgacatcgtttggctgatagacgggcagaaggtcgacgagcagttccctcaacacggcctcgtgaagcaggagggcaagctggcctccacacacagcgagctcaacatcacccagggccagtgggcgtccgaaaacacctacacctgccaggtcacttacaaagacatgatctttaaggaccaggcccgcaagtgcacagagtctgacccccgcggtgtgagcgtctacctgagcccgcccagccccctcgacctgtacgtctctaaatcgcccaagatcacctgcctggtggtggacctggccaacgtgcagggcttaagcctgaactggtcccgggagagcggggagcccctgcagaagcacacactggccaccagcgaacaatttaacaagacattctcggtcacgtccaccctgcctgtggacaccaccgactggatcgagggcgagacttacaagtgcaccgtgtcccacccagacctgcccagggaagtcgtgcgctccatcgccaaggcccctggcaagcgtttgtcccccgaggtctacgtgttcctgccgcctgaggaggaccagagctccaaggacaaggtcaccctcacctgcctgatccagaacttcttccccgcggacatctccgtacagtggctgcgtaacaatgtcctaatccagacagaccagcaagccaccacacggccccaaaaggccaatggccccaaccccgccttcttcgtcttcagccgcctagaggtcagccgggcggaatgggagcagaagaacaaatttgcctgcaaggtggtccacgaggcgctgtcccaaaggaccctccagaaagaggtgtccaaagaccctggtaaa

SEQ ID NO: 2 - protein, 424 aa, Equus caballus
  Domain (1)..(97): CH1 domain, IgE allotype a
VSKQAPLILPLAACCKDTKTTNITLGCLVKGYFPEPVTVTWDAGSLNRSTMTFPAVFDQTSGLYTTISRVVASGKWAKQKFTCNVVHSQETFNKTFNACIVTFTPPTVKLFHSSCDPGGDSHTTIQLLCLISDYTPGDIDIVWLIDGQKVDEQFPQHGLVKQEGKLASTHSELNITQGQWASENTYTCQVTYKDMIFKDQARKCTESDPRGVSVYLSPPSPLDLYVSKSPKITCLVVDLANVQGLSLNWSRESGEPLQKHTLATSEQFNKTFSVTSTLPVDTTDWIEGETYKCTVSHPDLPREVVRSIAKAPGKRLSPEVYVFLPPEEDQSSKDKVTLTCLIQNFFPADISVQWLRNNVLIQTDQQATTRPQKANGPNPAFFVFSRLEVSRAEWEQKNKFACKVVHEALSQRTLQKEVSKDPGK

SEQ ID NO: 3 - DNA, 1272 bp, Equus caballus
  Source (1)..(1272): cDNA based on mRNA from equine peropheral blood mononuclear cells
gtctccaagcaagccccattaatcttgcccttggctgcctgctgcaaagacaccaagactactaacatcacactgggctgcctggtcaagggctacttcccggagccagtgaccgtgacccgggatgcagggtcccttaaccggagcaccatgaccttccctgccgtctttgaccaaacctctggcctctacaccaccatcagcagggtggtcgcctcggggaagtgggccaagcagaagttcacctgcaacgtggtgcactcccaggagaccttcaacaagaccttcaacgcatgcatcgtgaccttcaccccacccaccgtgaagctcttccactcctcctgcgaccccggcggcgactcccataccaccatccagctcctgtgcctcatctccgactacacccctggcgacatcgacatcgtttggctgatagacgggcagaaggtcgacgagcagttccctcaacacggcctcgtgaagcaggagggcaagctggcctccacacacagcgagctcaacatcacccagggccagtgggcgtccgaaaacacctacacctgccaggtcacttacaaagacatgatctttaaggaccaggcccgcaagtgcacagagtctgacccccgcggtgtgagcgtctacctgagcccgcccagccccctcgacctgtacgtctctaaatcgcccaagatcacctgcctggtggtggacatggccaacgtgcagggcttaagcctgaactggtcccgggagagcggggagcccctgcagaagcacacactggccaccagcgaacaatttaacaagacattctcggtcacgtccaccctgcctgtggacaccaccgactggatcgagggcgagacttacaagtgcaccgtgtcccacccagacctgcccagggaagtcgtgcgctccatcgccaaggcccctggcaagcgtttgtcccccgaggtctacgtgttcctgccgcctgaggaggaccagagctccaaggacaaggtcaccctcacctgcctgatccagaacttcttccccgcggacatctccgtacagtggctgcgtaacaatgtcctaatccagacagaccagcaagccaccacacggccccaaaaggccaatggccccaaccccgccttcttcgtcttcagccgcctagaggtcagccgggcggaatgggagcagaagaacaaatttgcctgcaaggtggtccacgaggcgctgtcccaaaggaccctccagaaagaggtgtccaaagaccctggtaaa

SEQ ID NO: 4 - protein, 424 aa, Equus caballus
  Domain (1)..(97): CH1 domain, IgE allotype b
VSKQAPLILPLAACCKDTKTTNITLGCLVKGYFPEPVTVTRDAGSLNRSTMTFPAVFDQTSGLYTTISRVVASGKWAKQKFTCNVVHSQETFNKTFNACIVTFTPPTVKLFHSSCDPGGDSHTTIQLLCLISDYTPGDIDIVWLIDGQKVDEQFPQHGLVKQEGKLASTHSELNITQGQWASENTYTCQVTYKDMIFKDQARKCTESDPRGVSVYLSPPSPLDLYVSKSPKITCLVVDMANVQGLSLNWSRESGEPLQKHTLATSEQFNKTFSVTSTLPVDTTDWIEGETYKCTVSHPDLPREVVRSIAKAPGKRLSPEVYVFLPPEEDQSSKDKVTLTCLIQNFFPADISVQWLRNNVLIQTDQQATTRPQKANGPNPAFFVFSRLEVSRAEWEQKNKFACKVVHEALSQRTLQKEVSKDPGK

SEQ ID NO: 5 - DNA, 1601 bp, Equus caballus
  Source (1)..(1601): equine genomic DNA
gtctccaagcaagccccattaatcttgcccttggctgcctgctgcaaagacaccaagactactaacatcacactgggctgcctggtcaagggctacttcccggagccagtgaccgtgacctgggatgcagggtcccttaaccggagcaccatgaccttccctgccgtctttgaccaaacctctggcctctacaccaccatcagcagggtggtcgcctcggggaagtgggccaagcagaagttcacctgcaacgtggtgcactcccaggagaccttcaacaagaccttcaacggtgagccaggacggccccgcccgccctccagggggtgccgtcagaggaggaagggggggctggccaggagggcatcaccactgccggtgacagcctgggctgggacgtggcggcctgggctcagggaggccaacactgcgcccacccccaccgcccccagcatgcatcgtgaccttcaccccacccaccgtgaagctcttccactcctcctgcgaccccggcggcgactcccataccaccatccagctcctgtgcctcatctccgactacacccctggcgacatcgacatcgtttggctgatagacgggcagaaggtcgacgagcagttccctcaacacggcctcgtgaagcaggagggcaagctggcctccacacacagcgagctcaacatcacccagggccagtgggcgtccgaaaacacctacacctgccaggtcacttacaaagacatgatctttaaggaccaggcccgcaagtgcacaggtacagcccccgctcccccaaacatagacacccgacactcagggctcagaaaggagggcaggacacagcctcacacagccctcttcccaaaccacagagtctgacccccgcggtgtgagcgtctacctgagcccgcccagccccctcgacctgtacgtctctaaatcgcccaagatcacctgcctggtggtggacctggccaacgtgcagggcttaagcctgaactggtcccgggagagcggggagcccctgcagaagcacacactggccaccagcgaacaatttaacaagacattctcggtcacgtccaccctgcctgtggacaccaccgactggatcgagggcgagacttacaagtgcaccgtgtcccacccagacctgcccagggaagtcgtgcgctccatcgccaaggcccctggtgagccacgggccgaagggaggtgggcgggccccccggtggagactgggctgaccccatgcttgtccgtaggcaagcgtttgtcccccgaggtctacgtgttcctgccgcctgaggaggaccagagctccaaggacaaggtcaccctcacctgcctgatccagaacttcttccccgcggacatctccgtacagtggctgcgtaacaatgtcctaatccagacagaccagcaagccaccacacggccccaaaaggccaatggccccaaccccgccttcttcgtcttcagccgcctagaggtcagccgggcggaatgggagcagaagaacaaatttgcctgcaaggtggtccacgaggcgctgtcccaaaggaccctccagaaagaggtgtccaaagaccctggtaaa

SEQ ID NO: 6 - DNA, 21 bp, Artificial
  Oligonucleotide primer corresponding to 5′ end of equine C-epsilon 1 exon
gtctccaagcaagccccatta

SEQ ID NO: 7 - DNA, 32 bp, Artificial
  Oligonucleotide primer corresponding to antisense sequence of 3′ end of equine C-epsilon 4 exon with a Hind III cleavage site
tcgcaagctttaccagggtctttggacacctc

(19) United States
(12) Patent Application Publication
(10) Pub. No.: US 2004/0115764 A1
(43) Pub. Date: Jun. 17, 2004

(54) Desoxyribonucleic acids coding for the equine ige-allotype heavy chain constant region, the recombinant immunoglobulin obtained therewith and corresponding isotype-specific monoclonal antibodies and the utilization thereof

(76) Inventors: Wolfgang Leibold, Hannover (DE); Bettina Wagner, Wenniggen (DE); Andreas Raldbruch, Fresdorf (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/451,078

(22) PCT Filed: Dec. 20, 2001
(86) PCT No.: PCT/DE01/04810

(30) Foreign Application Priority Data
Dec. 21, 2000 (DE) 100 64 415.5

(51) Int. Cl. C12P 21/02; G01N 33/53; C07H 21/04; C12N 5/06; C07K 16/18; C07K 16/42

(52) U.S. Cl. 435/69.1; 435/320.1; 435/326; 530/388.1; 536/23.53; 530/387.2

(57) ABSTRACT

The invention relates to novel equine C_{ε}?a [symbol] and C_{ε}?b [symbol] genes coding for IgE-allotypes heavy chain constant regions and used in the production of IgE-isotype recombinant immunoglobulin. Said recombinant immunoglobulins represent valuable auxiliary agents for IgE-diagnosis, especially equine allergy diagnosis. With the aid of the recombinant IgEs, antibodies are obtained that can be used in allergy diagnosis, inter alia, in ELISA-based test kits.

Deoxyribonucleic acids which encode the constant region of the heavy chain of an equine IgE allotype, recombinant immunoglobulins obtained using them, and corresponding isotype-specific monoclonal antibodies and their use

The invention relates to DNA molecules for the constant region of the heavy chains of different equine IgE allotypes comprising newly found C_{ε}^{a} and C_{ε}^{b} genes, and recombinant class IgE immunoglobulins generated with the aid of these DNA sequences, which are valuable aids for equine diagnostics, in particular the diagnostics of equine allergies. The invention also relates to monoclonal anti-IgE antibodies which are raised with the aid of the recombinant immunoglobulins, and to their use in diagnostics and therapy.

Antibody responses in both desired reactions (protective antibodies owing to natural infection or inoculation) and undesired reactions, such as autoaggression reactions and allergies, play an important role in the organism.

Antibodies or immunoglobulins occur in the form of different classes and subclasses, hereinbelow referred to as isotypes. The isotype of the immunoglobulin decisively affects the functional properties which can be exerted by this molecule. In order to allow an assessment of immunological reactions to specific antigens, findings regarding an existing total antibody titer are not sufficient in a number of cases (for example in allergy diagnostics). Rather, a particular, antigen-specific isotype diagnosis is required in order to be able to assess the protective or else undesired functional properties of the antibodies which are generated in the context of an immune response.

In horses, for example, the isotypes IgM, IgGa, IgGb, IgGc, IgG(T), IgG(B), IgE and IgA were identified serologically (Lunn D. P., Holmes M. A., Schram B., Duffus W. P. H., (1995). Vet. Immunol. Immunopathol. 47:239-251; Butler J. E., (1998). Rev. Sci. Tech. Off. Int. Epiz. 17:43-70). However, studies into the equine genome have shown that a sixth IgG isotype exists in horses, since, correspondingly, six genes for these proteins exist (Wagner B., Overesch G., Sheoran A. S., Holmes M. A., Richards C., Leibold W., Radbruch A., (1998). Immunobiol. 199: 105-119).

The different function and pathogenetic importance of some equine isotypes is also mentioned in some studies: horses infected naturally with influenza viruses produced antigen-specific IgA, IgGₐ and IgG_{b} in the serum and the nasal secretions. These horses were protected from reinfection with influenza virus three months later. In contrast, inoculated horses reacted with the formation of IgG(T) and succumbed to the subsequent infection with the corresponding virus. While IgG(T) plays an important role in the neutralization of toxins (for example tetanus toxin), it is unable in the case of the abovementioned viral infection to bring about the decisive protective effector functions, such as complement activation or increasing phagocytotic activity (opsonizing effect).

As regards other undesired immune reactions, such as, for example, type I hypersensitivity, it is probable that, in horses as in other mammals, allergen-specific IgE antibodies play a decisive role for triggering the allergic reaction. As in humans, it is possible that in horses, too, an as yet uncharacterized IgG isotype is involved.

These few known examples demonstrate clearly the importance of isotype-specific diagnostics for a qualitative assessment of the antibody response. Compared with the determination of the overall antibody content, an isotype-specific diagnosis promises improved, clinically relevant findings regarding the protective, insufficient or pathogenic effects of the antibodies produced in the course of a particular immune response, not only for desired reactions of the immune system, such as protection from reinfection owing to natural infections, antibody responses triggered by inoculation, but also in the case of undesired immune reactions.

One of the aims of isotype-specific diagnostics—in horses and in other animals or indeed humans—consists in elucidating the protective or pathogenetic effects of the different immunoglobulin isotypes in relevant diseases or immune reactions and harnessing them for therapeutic purposes.

There is currently still a lack of reliable detection reagents, in particular for equine IgE, with the aid of which informative, isotype-specific -diagnostic methods can be developed. Monoclonal antibodies with high specificity for the isotype to be recognised while lacking cross-reactivity with other isotypes have proved particularly suitable. These properties of isotype-specific monoclonal antibodies are indispensable, in particular for detecting those immunoglobulin isotypes which occur in low concentrations only. Isotype-specific monoclonal antibodies can be employed in a large number of assay systems such as ELISA, flow-cytometric analyses, biochemical studies, cellular assays for the differentiation of B cells, functional assays (complement activation, phagocytotoxic activity, the release of mediators) and the like. Isotype-specific monoclonal antibodies already exist for some isotypes which are found in the serum in higher concentrations (IgM (Wagner B, Irienbusch S., Paetkau H., Sheoran A., Holmes M. A., Radbruch A., Leibold W., (1998). Immunobiol. 199:679), IgGa, b, c, (T) (Sheoran A. S., Lunn D. P., Holmes M. A., (1998). Vet. Immunol. Immunopathol. 62: 153-165), IgA), but no specific monoclonal detection reagents exist as yet for equines for the remaining IgG isotypes and also for IgE. The starting substance used for the production of the existing monoclonal antibodies was purified isotypes, which are found in the serum in sufficiently high concentrations. However, this method is difficult or indeed hopeless for physiologically rare isotypes, such as IgE.

A feasible route for the generation of monoclonal antibodies which are specific for equine IgE involves the production of recombinant reference substances which are very similar to, or identical with, natural equine IgE. To generate the equine recombinant IgE, it is first necessary to know the complete gene which encodes the equine IgE. However, chimeric immunoglobulins of, for example, murine light chains and equine heavy chains are also suitable for the intended end since the immunodominant epitopes, which are recognised specifically by antibodies, and the functional regions of the immunoglobulin are generally located on the constant domains of the heavy chains. Such chimeric constructs are known, for example, from “Generation of a recombinant mouse-human chimeric monoclonal antibody directed against human carcinoembryonic antigen”, Hardman et al., Int.J.Cancer 1989, 44 424-433, and “Expression of a recombinant sheep IgE gene” Clarke et al. in Immunological Investigations 23, 25-37 (1994).

Even though the complete mRNA, cDNA and corresponding amino acid sequences have been known for a number of years (NCBI sequence “U17041”-“Equus caballus Ig epsilon heavy chain mRNA, partial cds” (1994); NCBI sequence “U15150”-“Equus caballus IgE heavy chain mRNA, partial cds” (1996); “The complete cDNA and deduced amino acid sequence of equine IgE”, Navarro et al. in: Molecular Immunology 32, 1-8 (1995)), the production of satisfactory monoclonal antibodies which are specific for equine IgG has been unsuccessful up to the present invention.

Polyclonal antibodies directed against an equine IgE heavy-chain fragment expressed in E. coli are already known from “Chicken antibodies to a recombinant fragment of the equine immunoglobulin epsilon heavy-chain recognising native horse IgE”, Marti et al. in: Veterinary Immunology and Immunopathology 59 (1997), 253-270. This fragment comprises part of the CH3 and the CH4 domain of the heavy chain of an IgE allotype, that is to say it does not constitute a complete functional IgE molecule. It corresponds to natural IgE in the above-described region only with regard to the primary structure, that is to say the amino acid sequence. In contrast, complete immunoglobulins expressed in mammalian cells, such as the recombinant equine IgE described herein, have a high degree of homology with natural equine IgE even with regard to their tertiary structure. Furthermore, the high degree of glycosylation of natural equine IgE, which involves six N-glycosylation sites, is not found in bacterial expression systems, but has a pronounced effect on IgE structure and function. Thus, the N-glycosylation site at position N269 of the equine CH3 domain of the IgE^{a} and IgE^{b} sequences is involved in the binding to the equine F_{Cε} receptor (F_{Cε}RI) and is thus functionally important. In contrast, the recombinant IgE described by us in the present context, which is identical with or very similar to natural IgE in terms of structure and function, enables the development of highly-specific monoclonal anti-IgE antibodies.

These antibodies have a large number of advantages over polyclonal anti-IgE antibodies: monoclonal anti-IgE antibodies recognise a defined epitope in the region of the constant domains of the heavy chains of the IgE. As a rule, they have higher specificity and affinity for the corresponding epitope of the equine IgE, i.e. show no cross-reactivity with other proteins, in particular other equine immunoglobulin isotypes (IgM, IgG and the like; see 3.3 and 3.4) and show a higher sensitivity to equine IgE in diagnostic assay systems, which is approximately 1 ng IgE/ml serum in the case of the monoclonal anti-IgE antibodies described herein when detecting IgE by means of ELISA. The reason why the higher specificity and sensitivity is particularly important for the use of such monoclonal anti-IgE antibodies in diagnostics is that IgE, and in particular antigen-/allergen-specific IgE, is usually found only in very small amounts in the sample material (for example equine serum).

Owing to their epitope specificity, monoclonal anti-IgE antibodies additionally open up possibilities for therapy, for example of type I allergies in horses, in particular when these antibodies are capable of binding free IgE without simultaneously reactivating mast cells and/or basophile granulocytes via their receptor-bound IgE (see 3.5 and 3.6).

The object of the invention is therefore to provide recombinant immunoglobulins and specifically equine or equi-chimeric recombinant immunoglobulins which can act as reference substances in diagnostics. The invention furthermore encompasses the production of valuable isotype-specific monoclonal antibodies, in particular IgEs, for diagnostics and therapy with the aid of the IgE reference substance.

To achieve this aim, it is first necessary to identify and clone equine DNA regions which encode the constant domains of the equine IgE heavy chain and which are then fused in combination with complementing DNA segments for the variable domain of the heavy chain. Such an equine or chimeric DNA construct encodes, after it has been inserted into a suitable expression vector, a complete heavy chain of the equine or chimeric IgE. Complete immunoglobulins are then obtained by transfecting a cell line which secretes light immunoglobulin chains with such an expression vector.

The resulting recombinant IgE was used for raising monoclonal IgE-specific detection antibodies, and thus constitutes the basis for informative IgE diagnostics in horses. Moreover, the monoclonal anti-IgE antibodies may also be exploited for therapeutic approaches.

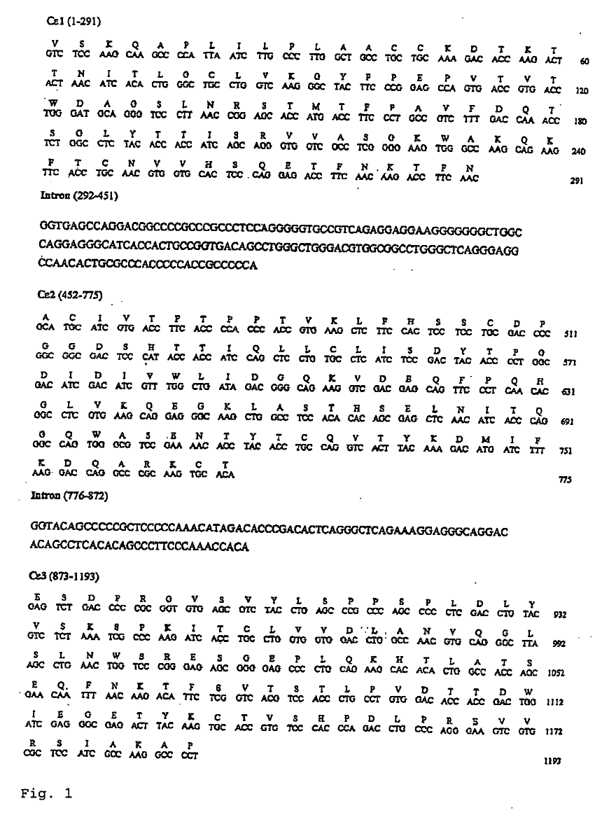

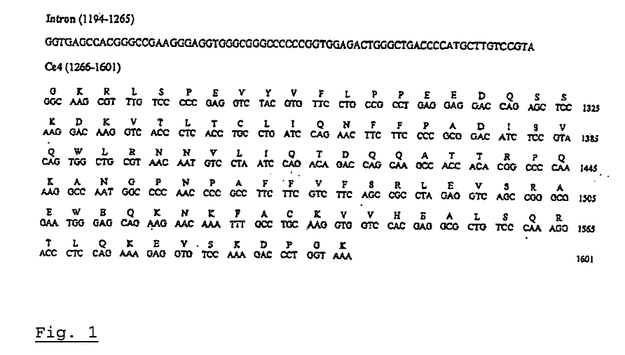

In accordance with the invention, a C_{ε} gene is used for preparing the recombinant IgEs, i.e. an mRNA which encodes the constant region of the heavy chain of an equine IgE allotype is obtained from peripheral horse blood and transcribed into a C_{ε}-cDNA as described in the examples. Two allelic forms which encode two different equine IgE allotypes were found and referred to as C_{ε}^{a} and C_{ε}^{b}. The sequences of these novel, allotype-specific C_{ε} genes, together with the corresponding amino acid sequence, are indicated in FIG. 1. The sequence of the C_{ε}^{a} gene, the C_{ε}^{b} gene and the corresponding amino acids are also mentioned in the sequence listing.

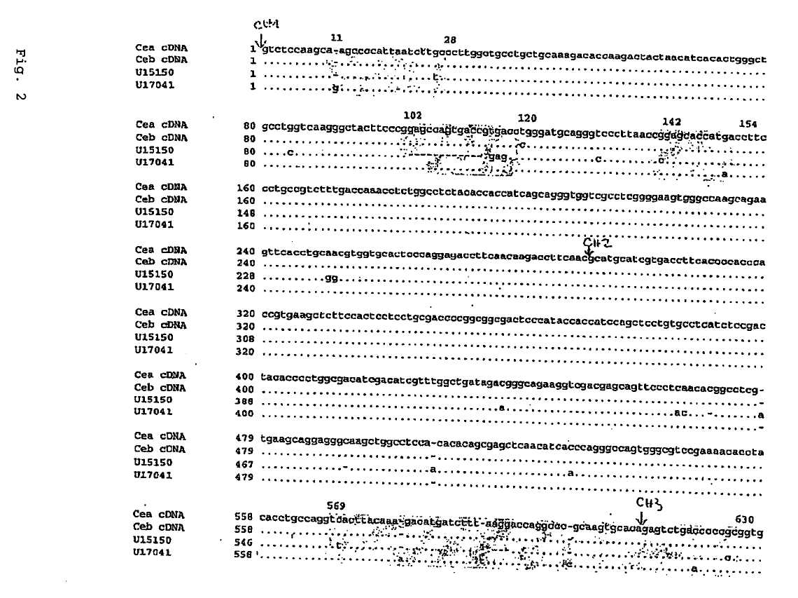

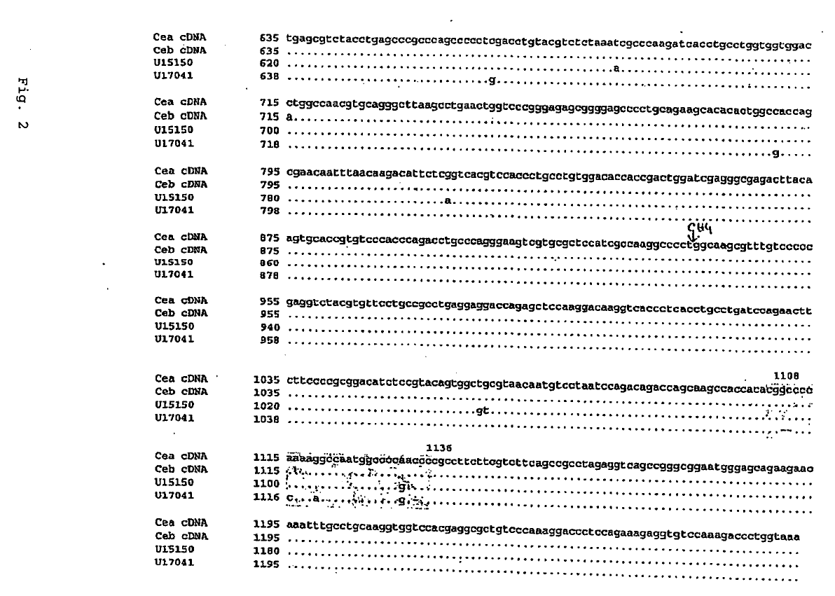

The equine C_{ε}^{a} and C_{ε}^{b} sequences which encode the constant region of the heavy chain of an equine IgE allotype and which can be used for the purposes of the invention are distinguished by the fact that they agree at least in the region from T569 to C630 with the sequences as shown in Seq.ID 1 and Seq.ID 3 indicated in FIG. 1, as is stated in FIG. 2. Functionally important regions are shown against a dark background in FIG. 2. As has been shown experimentally, and in particular when using these genes, it is possible to obtain recombinant IgE which is identical structurally and functionally with natural IgE and with which, in turn, antibodies can be developed which are specific for equine IgE and thus valuable in the diagnostics of equine allergies. The functionality of the IgEs generated with the C_{ε}^{a} and C_{ε}^{b} genes according to the invention, which has been found in this context, is not a matter of course since mutations in functional regions may lead inter alia to modifications in the tertiary structure or binding capacity of the IgE so that antibodies generated with recombinant IgE which is not largely identical with natural IgE as regards its tertiary structure, are not, or not optimally, specific for natural equine IgE. In accordance with the invention, in contrast, antibodies are obtained which bind to natural equine IgE with high specificity, as will be demonstrated hereinbelow. Thus, monoclonal antibodies which are specific for natural equine IgE have been produced successfully for the first time.

Thus, it is possible for the first time, with the aid of the novel C_{ε}^{a} and C_{ε}^{b} genes, to generate functional recombinant immunoglobulins with the aid of which antibodies which are specific for equine IgE can be obtained.

Instead of the abovementioned C_{ε}^{a} and C_{ε}^{b} genes, it is also possible to use equivalent homologous sequences which lead to corresponding functional immunoglobulins, but with the exception of the nucleotide sequence for the C_{ε}^{c} and C_{ε}^{d} gene (NCBI sequences U15150 and U17041).

The newly found C_{ε} genes agree in that they have at least 55% homology with a corresponding human sequence.

The homology of the equine C_{ε} sequences to the human one is relatively low. The homology levels found for the novel allotypes were: C_{ε}^{a}:56.4%; C_{ε}^{b}:56.0%, while the level of homology of the C_{ε} sequence NCBI U15150 (see above, C_{ε}^{c}, Navarro P., Barbis D. P., Antczak D., Butler J. E., 1995, Mol. Immunol. 32:1-87) is only 54%. The percentage of conserved amino acid sequence regions in comparison with human IgE is thus relatively high in the case of the newly found equine IgE allotypes. The sequence differences between the equine C_{ε} alleles can also be demonstrated with the aid of restriction fragment length polymorphisms (RFLPs). The following holds true for the restriction enzymes StuI and SmaI:

C_{ε}^{a}: one SmaI site (position 751)

C_{ε}^{b}: two SmaI sites (positions 121 and 751)

C_{ε}^{c}: (published in “Navarro”): one StuI site (position 493), two SmaI sites (positions 107 and 790; according to published sequence)

C_{ε}^{d}(U17041): one SmaI site (position 808)

Chimeric recombinant immunoglobulins are prepared in a manner known per se. One possible method is described by Clarke et al. (loc.cit.). To carry out this method, C_{ε}^{a} or C_{ε}^{b} DNA can be cloned into a cloning cassette of a eukaryotic expression vector. The V_{H} gene, for example the V_{H}186.2 cDNA (GenBank Acc.No. J00529; Bothwell A. L. M., Paskind M., Reth M., Imanishi-Kari T., Rajewsky K., Baltimore D., (1981). Cell 24: 625-637) can subsequently be cloned into the expression vector 5′ of the Cε gene. The C_{ε}-cDNA can be excised whenever desired from an expression vector thus obtained and can be replaced by any desired heavy-chain gene. In this manner, further recombinant immunoglobulins can be obtained with the aid of this construct according to the invention by exchange of the C_{ε} gene or gene fragments.

Monoclonal isotype-specific anti-equine-IgE antibodies are raised by standard methods via the immunization of experimental animals with the recombinant equi-murine IgE. The result is monoclonal antibodies which are specific for equine IgE which is characterized by the respective C_{ε} allele used for producing the recombinant protein. Such monoclonal antibodies, which in exceptional cases can recognise the murine components of the recombinant chimeric IgE, can be eliminated for example using the assay described in the examples in Section 3.1. In most cases, however, epitopes on the equine heavy-chain region (which is the rule in the isotypes studied in the present context) serve for the recognition of these IgEs by monoclonal anti-IgE antibodies.

The recombinant chimeric DNA used for the purposes of the present invention gives rise to immunoglobulin molecules which correspond largely to natural ones, since the monoclonal antibodies obtained with the aid of the reference substance according to the invention (IgE) show a high degree of isotype specificity for both the reference substance and natural equine IgE (see hereinbelow, Table 1). Thus, the method according to the invention provides a good yield of very advantageous Ig products which resemble natural ones, and these Ig products can be used as reference substances and for the production of highly specific monoclonal antibodies.

The sequences according to the invention, which are shown in the sequence listing, are represented in relation to each other in FIG. 1. FIG. 2 shows the sequences of the C_{ε}^{a}-cDNA (Cea-cDNA) and of the C_{ε}^{b}-cDNA (Ceb-cDNA) in comparison with the known sequences. U15150 (NCBI, Equus caballus Ig epsilon heavy chain mRNA; partial cds, Navarro, P., Barbis, D. P., Antczak, D. and Butler, J. E.) and U17041 (NCBI, Equus caballus IgE heavy chain mRNA, Watson, J. L., Wilson, L. K. and Gershwin, L. J.):

FIG. 1: Equine genomic Cε^{a} nucleotide and amino acid sequence

The two nucleotide substitutions in Cε1 (121 T→C) and the Cε3 exon (972 C→A) of the Cε^{a} and Cε^{b} alleles are shown against a gray background. Both base substitutions bring about modifications in the amino acid sequence in the CH1 (41 W→R) and CH3 domains (239 L→M) of the resulting IgE allotypes.

FIG. 2: Sequence alignment between Cea-cDNA (Seq.ID1), Ceb-cDNA (Seq.ID3) and NCBI sequence U15150 and NCBI sequence U17041.

The invention is described in more detail hereinbelow with reference to a practical example:

Description of the isolation of novel equine Cε sequences (Cε^{a} and Cε^{b}), their use for producing a functional, recombinant equine IgE, and th first development of monoclonal antibodies directed against equine IgE:

1. Isolation of Equine Cε cDNA and its use for Expressing Recombinant Equine IgE

1.1. Obtaining the cDNA for the Constant Region of the Equine IgE Heavy Chain (Cε)

DNA primers were synthesized according to the published Cε sequence (Navarro P., Barbis D. P., Antczak D., Butler J. E., (1995). Mol. Immunol. 32: 1-87): 5′ GTCTCCAAGCAAGCCCCATTA 3′-corresponds to the 5′ end of the equine Cε1 exon and 5′ TCGCAAGCTTTACCAGGGTCTTTGGACACCTC 3′-corresponds to the antisense sequence of the 3′ end of the Cε4 exon and contains a Hind III cleavage site.

Following standard methods, mononuclear cells of the peripheral blood of a horse were used for obtaining the total RNA (RNeasy-Kit, Quiagen, Hilden, FRG). The equine RNA was transcribed into cDNA by means of a reverse-transcriptase reaction using an oligo(dT)₁₅ primer (Promega, Mannheim, FRG) and SuperScript II reverse transcriptase (Life Technologies, Karlsruhe, FRG). Using this cDNA and the above-described primers, an equine Cε cDNA sequence was amplified by means of polymerase chain reaction. To this end, 1 μl of cDNA was mixed with a reaction mixture consisting of 4 mmol MgCl₂, 200 μmol of each DNTP (DATP, dTTP, dCTP, dGTP; Promega, Mannheim, FRG), 0.2 pmol of each primer (Life Technologies, Karlsruhe, FRG) and 1.25 U Pfu DNA polymerase in 1× Pfu DNA polymerase buffer (Promega, Mannheim, FRG) and amplified in a thermocycler (Biometra, Göttingen, FRG). In addition, a genomic Cε gene which we had isolated from an equine genomic gene library and cloned (Wagner B., Siebenkotten G., Leibold W., Radbruch A, (1997). Vet. Immunol. Immunopathol. 60: 1-13) was used as template and likewise amplified in this polymerase chain reaction. The two Cε sequences were sequenced (SEQ LAB, Gottingen, FRG) and, even though they are derived from different, non-related horses, show 100% nucleotide sequence homology within the coding regions. However, the sequence homology with the Cε sequences which have already been published amounts to only 96% (GenBank Acc.No. U15150; Navarro P., Barbis D. P., Antczak D., Butler J. E., (1995). Mol. Immunol. 32: 1-87) and 98% (GenBank Acc.No. U17041; Watson J. L., Pettigrew H. D., Wilson L. K., Gershwin L. J., (1997). J. Vet. Allergy Clin. Immunol. 5: 135-142). These differences between the Cε sequences determined by ourselves (FIG. 1, Cε^{a}) and those which have been published earlier allow the conclusion that different Cε alleles exist in horses. Cε alleles were identified in a substantial number of non-related horses by means of restriction analysis with the restriction endonucleases Sma I and Stu I, which, owing to the sequence differences, have different cleavage sites within the Cε cDNA. In this process, a further Cε allele (Cε^{b}) was identified, and this allele deviates from the Cε^{a}, which had been sequenced by ourselves, in two bases. Both base substitutions in the Cε^{b} allele also result in amino acid substitutions at the corresponding positions (FIG. 1), i.e. the two new alleles Cε^{a} and Cε^{b}, like the Cε alleles which are known to date (U15150, referred to as Cε^{c}, and U17041, referred to as Cε^{d}), encode different IgE allotypes. The resulting modifications in the derived amino acid sequences of the four IgE allotypes which have been identified to date may also result in functional modifications, such as, for example, in a different binding behavior at FCε receptors and/or modifications in the ability of bringing about the release of inflammatory mediators from mast cells. These functional differences may play a role in particular in the development of type I allergies.

1.2. Expression of Equine Recombinant IgEs

The method used in this context for expressing recombinant immunoglobulins is known (Oi V. T., Morrison S. L., Herzenberg L. A., Berg P., (1983), Proc. Natl. Acad. Sci. USA 80: 825-829; Knight K. L., Suter M., Becker R. S., (1988). J. Immunol. 140: 3654-3659; Clarke R. A., Beh K. J., (1994). Immunol. Invest. 23: 25-37). The principle of the procedure for expressing the complete equine recombinant IgE which has been generated for the first time will be summarized hereinbelow:

To produce a recombinant equine IgE, the above-described equine Cε^{b} cDNA and the murine V_{H}186.2 cDNA (GenBank Acc.No. J00529; Bothwell A. L. M., Paskind M., Reth M., Imanishi-Kari T., Rajewsky K., Baltimore, D., (1981). Cell 24: 625-637), which together encode the chimeric heavy immunoglobulin chain of IgE, were cloned into a eukaryotic expression vector. This construct was used to transfect the murine myeloma cell line J558L which produces murine light λ chains (Oi V. T., Morrison S. L., Herzenberg L. A., Berg P., (1983). Proc. Natl. Acad. Sci. USA 80: 825-829). The cells which secreted complete IgE immunoglobulins were subsequently selected. Light chains from the J558L cell line together with heavy chains containing the V_{H}186.2 gene product form antibodies with a defined antigen specificity for 4-(hydroxy-3-nitrophenyl)acetyl (NP), in this case NP-specific equine IgE. Protein-biochemical analyses of the expressed protein have demonstrated that this recombinant IgE has high structural similarity with native equine IgE. Moreover, the recombinant protein binds to the FCCRI of mast cells and basophile granulocytes and is capable of mediating a release of inflammatory mediators from these cells in vitro and in vivo, i.e. it also corresponds to native equine IgE with regard to the functional properties.

2. Raising IgE-Specific Monoclonal Antibodies (Anti-Equine IgE)

2.1. Immunization of Mice

Female BALB/C mice were immunized with recombinant IgE. The purified equine NP-specific IgE (NP-IgE), was employed in a total amount of 2.5 μg at the first immunization and 1.25 μg for all further immunizations. For the first (day 0), the second (day 14) and the third immunization (day 21), the protein was mixed with Gerbu Adjuvanz MM (Gerbu Biotechnik, Gaiberg, FRG) following the manufacturer's instructions. For the further immunizations on days 28, 29 and 30, the NP-IgE was applied in PBS without added adjuvant. All injections were given intraperitoneally. Cell fusion was performed on day 31.

2.2. Raising Monoclonal Antibodies

On day 31, one mouse whose NP-IgE serum titer had previously been studied (ELISA see 2.3.1.) was sacrificed, the spleen was removed under sterile conditions, and the spleen cells were plated out carefully. The spleen cells were taken up in Hybridoma SFM medium (Life Technologies, Karlsruhe, FRG), counted and mixed 1:2 with murine X63-Ag8.653 myeloma cells (Kearney J. F., Radbruch A., Liesegang B., Rajewsky K., (1979). J. Immunol. 123: 1548-1550). Following centrifugation and removal of the supernatant, the cells were resuspended carefully and treated slowly with 1.5 ml polyethylene glycol 1500 (Boehringer, Mannheim, FRG) which had been warmed to 37° C. After incubation for 1 minute at 37° C., 20 ml of Hybridoma SFM medium were slowly added dropwise to dilute the polyethylene glycol (1 ml over 1 minute, 3 ml over 1 minute, 16 ml over 1 minute). Following centrifugation and removal of the supernatant, the cell pellet was resuspended carefully in 200 ml of Hybridoma SFM supplemented with HAT media supplement (Sigma, Steinheim; FRG), 10% (v/v) Myclone° FKS (Life Technologies, Karlsruhe, FRG), 100 IU/ml penicillin, 100 μg/ml streptomycin (PAN Biotech, Aidenbach, FRG) and 4 U/ml human recombinant IL6. This cell suspension was plated into 24-well cell culture plates. After 7-10 days, individual clones were visible. They were picked from the 24-well plates and transferred into 96-well plates. After a further 2-3 days, the supernatants of these 96-well plates were tested in an ELISA assay for anti-IgE-specific antibodies. Positive clones were characterized further (see 3.) and recloned once or twice. The HAT supplement in the medium was replaced after two weeks by HT supplement (Sigma, Steinheim, FRG). After a further 3-4 weeks, the cells were weaned onto Hybridoma SFM medium without further selection additives and without human recombinant IL6.

3. Detecting the IgE Specificity of the Monoclonal Antibodies

The IgE specificity of the (in total) 18 monoclonal antibodies (Table 1) was detected by standard methods which were modified in a suitable manner for this purpose. Monoclonal antibodies which specifically recognised the heavy chain of the recombinant equi-murine NP-IgE were detected in ELISA assays (see 3.1.). The ability of the monoclonal antibodies to recognise not only the recombinant protein, but also native equine IgE, was verified by SDS-PAGE (see 3.2.) and membrane immunofluorescence (see 3.3.). The specificity of the monoclonal anti-IgE antibodies for native equine IgE, and the lack of reaction with all other equine immunoglobulins available, were verified in an isotype-specific ELISA (see 3.4.). The specificity of the anti-IgE antibodies for various epitopes of the equine IgE was detected in an inhibition ELISA (see 3.5.).

3.1. Detection of NP-IgE-Specific Monoclonal Antibodies

Following cell fusion, the supernatants of the clones which had grown were first analyzed for the presence of specific antibodies which react with the recombinant IgE. An ELISA was used for detecting monoclonal antibodies which recognize the NP-IgE heavy chain. The ELISA plates (Nunc, Wiesbaden, FRG) were coated with NP derivative 4-(hydroxy-3-indo-5-nitrophenyl)acetyl (NIP) conjugated with bovine serum albumin (BSA) (NIP₁₅-BSA; Biosearch Technologies, Navato, Calif., USA) in a concentration of 5 μg/ml in carbonate buffer (15 mmol Na₂CO₃, 35 mmol NaHCO₃, pH 9.6). After the plates had been washed with phosphate buffer (2.5 mmol NaH₂PO₄, 7.5 mmol Na₂HPO₄, 145 mmol NaCl, 0.1% (v/v) Tween 20, pH 7.2), they were incubated with NP-IgE, which binds to the NIP-BSA-coated plate. After a further washing step, the supernatants from the 96-well plates of the cell fusion were then applied to the plate thus prepared. If NP-IgE-specific monoclonal antibodies were present, they were bound in this step to the ELISA plate and, after a further washing step, detected using a peroxidase-conjugated polyclonal anti-mouse-IgG antibody (Dianova, Hamburg, FRG). After addition of substrate solution (33.3 mmol citric acid, 66.7 mmol NaH₂PO₄, pH 5.0), freshly treated with 130 pg/ml 3,3′,5,5′-tetramethylbenzidine (TMB, Sigma, Steinheim, FRG) and 0.01% (v/v) hydrogen peroxide (Sigma, Steinheim, FRG)), the anti-IgE-antibody producing clones were identified by the color reaction which had taken place.

As a distinction from monoclonal antibodies which recognize the murine portions of the NP-IgE, the supernatants which were positive in the first assay were additionally checked on a plate which was coated with NIP-BSA and subsequently incubated with murine NP-IgD. Monoclonal antibodies which specifically recognized the equine IgE heavy chain reacted only with NP-IgE, but not with NP-IgD.

3.2. Biochemical Detection of IgE in Equine Serum

IgE is found in the serum in small concentrations only and has a short half-life. However, in particular in allergic patients, serum IgE levels may rise drastically. IgE was detected using the monoclonal anti-IgE antibodies after the equine serum had been separated by sodium dodecyl sulfate/polyacrylamide gel electrophoresis (SDS-PAGE). To this end, the equine sera were treated with SDS sample buffer (62.5 mmol Tris, 10% (v/v) glycerol, 2% (w/v) SDS, 0.1% (w/v) bromphenol Blue, pH 6.8), pH 6.8) and separated under nonreducing conditions in a 7.5% SDS gel in a Mini Protean II chamber (Bio-Rad Laboratories, Munich, FRG). These proteins were transferred from the gel to a polyvinylidene difluoride membrane by Western Blotting which was incubated with the monoclonal anti-IgE antibodies after the free binding sites had been blocked with 1% (w/v) gelatin. The monoclonal anti-IgE antibodies identified an approx. 220 000 Dalton protein in the equine serum, which corresponds to the molecular weight of equine IgE. The protein identified by the monoclonal antibodies was not detectable in the serum of all of the horses studied; however, in particular horses with clinical allergic symptoms usually also showed a pronounced IgE band in the serum.

The immunoglobulins in the equine serum were separated into their light and heavy chains under reducing conditions by treating the SDS sample buffer with 5% (v/v) 2-mercaptoethanol. One monoclonal anti-IgE antibody (αIgE-176) also recognized the isolated equine IgE heavy chain with a relative molecular weight of 76 000 Daltons. Accordingly, the αIgE-176 antibody recognizes a different epitope of the equine IgE than the remaining monoclonal antibodies, which only recognize the unreduced IgE (see 3.5.). In contrast, the IgE epitope recognized by αIgE-176 is also present on the isolated IgE heavy chain.

3.3. Labeling IgE on Equine Blood Leukocytes

IgE can be bound at the surface of certain blood leukocytes by what are known as Fc receptors (in the present case FcεRI or FcεRII). FcεRI can be expressed by basophile and eosinophile granulocytes, while FccRII can be expressed by some of the monocytes, B cells and eosinophile granulocytes. Free serum IgE can be bound to the cells via these receptors, so that it can be detected at the cell surface by fluorochrome-coupled antibodies (membrane immunofluorescence).

The equine blood leukocytes were obtained from anticoagulant-treated whole blood of various horses. To this end, the leukocyte-rich plasma above the erythrocyte sediment was obtained after approximately 30 minutes of spontaneous sedimentation and then centrifuged; thereafter, the leukocytes were washed 2× with PBS in order to remove the thrombocytes (80×g, 5 min). Thereafter, the leukocytes were taken up in PBS/BSA (PBS supplemented with 0.5% (w/v) bovine serum albumin and 0.02% (w/v) sodium azide) and placed on ice. 5×10⁶ aliquots of equine leukocytes were incubated on ice for 10 minutes in 10 μl of PBS/BSA in a tube containing 30 μl of the monoclonal anti-IgE antibodies (1:2 in PBS/BSA). As a control, 5×10⁶ aliquots of equine leukocytes were incubated under identical conditions with an irrelevant murine monoclonal IgG1 antibody (isotype control). After the cell samples had been washed once with cold PBS/BSA, they were all incubated for 5 minutes on ice together with a phycoerythrin-conjugated anti-mouse IgG antibody (Dianova, Hamburg, FRG), washed again and, after addition of propidium-iodide-containing PBS, measured in a flow cytometer. The amount of surface-IgE-positive cells was 1.28±0.52% in the case of the adult horses studied and differed highly significantly (p<0.001) from the isotype control 0.02±0.02%.

The IgE-positive cells of adult horses were isolated by magnetic cell sorting and studied under the microscope and by flow cytometry. The cell fraction which can be isolated by the monoclonal anti-IgE antibodies which were developed consists to approximately 30% of basophile granulocytes which have bound IgE via their FccRI, to approx. 68% of mononuclear cells (lymphocytes, lymphoblasts and monocytes) which are capable of binding IgE complexes with their FcεRII, and to a minor extent of approx. 2% of other cells (for example eosinophile granulocytes). These studies demonstrate that the monoclonal anti-IgE antibodies are capable of recognizing equine IgE not only in its native form (see 3.2.), but also when bound to Fcε receptors.

3.4. Isotype-Specific ELISA

To detect the IgE specificity of monoclonal antibodies, the ELISA plates were coated with a polyclonal anti-horse IgG(H+L) antibody (Dianova, Hamburg, FRG) and subsequently incubated with various equine reference immunoglobulins (IgM, IgGa, IgGb, IgG(T) light chains, purified serum IgE). In the next step, the monoclonal anti-IgE antibodies were incubated with in each case all of these reference proteins, and the anti-IgE binding was then visualized by means of a peroxidase-conjugated anti-mouse IgG antibody and the subsequent substrate reaction. Binding of the anti-IgE antibodies to the purified serum IgE was detected, but not to the other equine immunoglobulins.

3.5. Inhibition ELISA for Identifying Different Epitope Specificities of the Anti-IgE Antibodies.

The inhibition ELISA made possible the identification of different IgE epitopes which are recognized and bound by the various monoclonal anti-IgE antibodies. To this end, the ELISA plates were coated with NIP₁₅-BSA and subsequently incubated with recombinant IgE. Then, the 18 different monoclonal anti-IgE antibodies were applied to the plates thus coated with recombinant IgE. During the incubation time, the antibodies had a chance to bind to their respective specific epitopes of the equine recombinant IgE. In the next step, the biotinylated αIgE-134 antibody, which was only capable of binding with the recombinant IgE if the epitopes which this αIgE-134 antibody recognizes on the recombinant IgE were still freely accessible, i.e. not blocked by one of the anti-IgE antibodies in the previous step, was added. Binding of the biotinylated αIgE-134 antibody was then visualized using streptavidine-peroxidase and a final substrate reaction. The epitopes of the recombinant IgE which are recognized by the monoclonal antibodies αIgE-22, αIgE-41, αIgE-132 and αIgE-176 did not inhibit the binding of the biotinylated αIgE-134 antibody, i.e. these anti-IgE antibodies recognize different epitopes of the recombinant IgE.

3.6. Capability of Basophile Granulocytes of being Activated by the Monoclonal Anti-IgE Antibodies

The anti-IgE antibodies αIgE-41, αIgE-132, αIgE-134 and αIgE-176 were studied for their ability to release mediators from equine basophile granulocytes. To this end, these monoclonal antibodies were employed in a histamine release assay which has already been described (Kaul, S., 1998. Typ I Allergien beim Pferd: Prinzipielle Entwicklung eines funktionellen in vitro Nachweises [Type I Allergies in horses: principle of the development of a functional in-vitro assay] PhD thesis, Veterinary School Hanover)), in which the ability of the various monoclonal antibodies to release histamine from equine blood basophiles is measured in relation to the maximum and spontaneous release of histamine from these cells. The induction of histamine release was only achieved with the antibody αIgE-134.

Data on the characterization of monoclonal anti-IgE antibodies are compiled in table 1.

TABLE 1
Characterization of monoclonal anti-IgE antibodies (αIgE)

| | | Immunoblot | | | Binding to other | Inhibition ELISA (see 3.5.) | |
|---|---|---|---|---|---|---|---|
| | ELISA (see 3.1) | (see 3.2.) | | Flow cytometry | equine isotypes | IgE epitopes | |
| αIgE | NP-IgE⁺/NP-IgD⁻ | IgE(−ME) | IgE(+ME) | (see 3.3.) | as IgE (see 3.4.) | ≠αIgE−134 | =αIgE−134 |
| 22 | + | + | − | + | − | + | |
| 28 | + | + | − | + | − | | + |
| 41 | + | + | − | + | − | + | |
| 43 | + | + | − | + | − | | + |
| 112 | + | + | − | + | − | | + |
| 119 | + | + | − | + | − | | + |
| 120 | + | + | − | + | − | | + |
| 132 | + | (+) | − | − | − | + | |
| 134 | + | + | − | + | − | | + |
| 150 | + | + | − | + | − | | + |
| 151 | + | + | − | + | − | | + |
| 176 | + | + | + | + | − | + | |
| 336 | + | + | − | + | − | | + |
| 521 | + | + | − | + | − | | + |
| 522 | + | + | − | + | − | | + |
| 580 | + | + | − | + | − | | + |
| 594 | + | + | − | + | − | | + |
| 672 | + | + | − | + | − | | + |

+ = positive;
− = negative;
(+) = positive only with recombinant IgE

We claim:

1. A DNA whose nucleotide sequence encodes the constant region of the heavy chain of an equine IgE allotype and which agrees at least in the region from T569 to C630 with the equine C_{ε}^{a} sequence as shown in Seq.ID1 or the equine C_{ε}^{b} sequence as shown in Seq.ID3.

2. A DNA as claimed in claim 1, characterized in that it comprises the C_{ε}^{a} gene as claimed in Seq.ID1 or the C_{ε}^{b} gene as shown in Seq.ID3 or functional regions of these.

3. A recombinant class IgE immunoglobulin, characterized in that it comprises constant regions of heavy chains of an equine IgE allotype which are encoded by a DNA sequence as claimed in claim 1 or 2.

4. A recombinant immunoglobulin as claimed in claim 3, characterized in that it comprises variable regions (V_{H}) of the heavy immunoglobulin chains of a different species, preferably a murine V_{H} region.

5. A recombinant immunoglobulin as claimed in claim 3 or 4, characterized in that it comprises murine light chains.

6. A recombinant immunoglobulin as claimed in one of claims 3 to 5, characterized in that it is antigen-specific and preferably comprises NP-specific variable regions.

7. A monoclonal antibody, characterized in that it is specific for equine immunoglobulin E.

8. A monoclonal antibody, characterized in that it is IgE-specific and can be obtained by immunizing laboratory animals with a recombinant immunoglobulin as claimed in one of claims 3 to 6.

9. The use of an antibody as claimed in claim 7 or 8 for allergy diagnostics in horses.

10. A test kit for allergy diagnostics, characterized in that it comprises antibodies as claimed in claim 7 or 8.
